(19) United States
(12) Patent Application Publication
(10) Pub. No.: US 2005/0245837 A1
(43) Pub. Date: Nov. 3, 2005

(54) Mouthpiece for use in a spirometer

(75) Inventors: Vadim I. Pougatchev, Poulsbo, WA (US); Yevgeniy N. Zhirnov, Poulsbo, WA (US); Evgueni N. Gribkov, Kingston, WA (US); Mark Ferris, Silverdale, WA (US)

Correspondence Address: Winstead Sechrest & Minick P.C., P.O. Box 50784, Dallas, TX, 75201, US

(73) Assignee: MedPond, LLC, Austin, TX (US)

(21) Appl. No.: 10/833,361

(22) Filed: Apr. 28, 2004

(51) Int. Cl. A61B 5/08

(52) U.S. Cl. 600/538

(57) ABSTRACT

The invention entails a mouthpiece for use in a spirometer. In one embodiment of the invention, the mouthpiece comprises a tube forming a conduit between an upstream end and a substantially closed downstream end. An opening is formed through the tube and is proximate to the downstream end of the tube. The mouthpiece also comprises a resistive element that is positioned substantially across the tube opening. In addition, the mouthpiece comprises an outer sleeve that is slide along the exterior of the tube. The outer sleeve may be slid along the tube thereby covering portions of the tube opening in varying amounts. Thus, the outer sleeve may be slid into one position wherein the tube opening is uncovered. The outer sleeve may then be advanced into a closed position wherein the tube opening is substantially sealed. Many partially closed positions exist between the open and closed positions thereby allowing for variable amounts of resistance to air flow. To clearly indicate to the user how far the outer sleeve has been advanced, markings may be made on the outer sleeve or tube indicating, for example, the first tube opening is fifty percent occluded.

### BACKGROUND OF THE INVENTION

1. Field of the Invention

The present invention relates to the field of measuring air pressure, and related characteristics, associated with air discharged from a patient's lungs during physiological testing, including spirometric and heart rate variability studies. More specifically, the invention is related to an apparatus that the patient may breath into during such studies whereby the apparatus provides resistance to air flow and allows for pressure readings to be taken.

2. Description of the Related Art

Spirometry concerns methods for studying pulmonary ventilation. In a typical spirometry study, a patient blows into a spirometer which includes a mouthpiece with a known resistance. The spirometer allows a physician to measure the patient's respiratory air pressure, flow rate and volume. A physician can then use those results to obtain respiratory-related physiological values such as tidal volume, inspiratory reserve volume, expiratory reserve volume and residual volume. In addition to studying the respiratory system, a physician may use a spirometer to study a patient's autonomic nervous system (ANS). A patient's ANS regulates many organs including the heart. In a study aptly named a “heart rate variability” (HRV) study, a physician can evaluate the ability of the subject's ANS to regulate the heart by monitoring how the patient's heart rate varies (i.e., how the heart is regulated) in response to certain conditions, such as heavy breathing. HRV studies may utilize spirometers because a patient may need to breath at a certain rate and a certain pressure to properly tax and evaluate the respiratory and nervous systems. Spirometers help measure these breathing rates and pressures by recording the patient's respiration. In short, spirometers have medical utility for monitoring both the respiratory and nervous systems.

A spirometer typically includes a mouthpiece that is connected, often through a length of tubing, to a pressure transducer or recording device. Taking the expiration cycle of respiration as an example, after the patient blows into a mouthpiece his expired air puts pressure on air already present within the length of tubing. This “pressure wave” is transmitted to the pressure transducer which is connected to the tubing. The transducer converts this mechanical pressure wave into an electronic signal. The electronic signal is then amplified and filtered before being digitized via an analog-to-digital converter. A system processor then facilitates transfer of the electronic signal to memory. The processor may also facilitate the calculation of various physiological measurements from the signal.

Focusing on the spirometer's mouthpiece in particular, as a patient breathes through the mouthpiece of, for example, a differential pressure spirometer, the air flow encounters a “resistive element” which provides resistance to the air flow. The resistive element may be nothing more than a breathable, mesh disc placed within the tube. The resistive element may also be a series of hinged windows (e.g., U.S. Pat. No. 5,743,270) or even a parabolic form that deflects air flow through mesh-covered panels (e.g., U.S. Pat. No. 4,905,709). Using the mesh disc resistive element as an example, the patient's air flows across the element. Due to the resistive nature of the element, the air pressure is higher “upstream” of the element than it is “downstream” of the element in a manner analogous to a dam in a river. The difference in the pressure upstream of the resistive element and the pressure downstream of the resistive element is proportional to the air flow through the tube. In other words, a patient that breathes forcibly through the mouthpiece, and across the resistive element, will have a greater “pressure differential” and air flow than a patient that breathes meekly through the mouthpiece. To help monitor the pressure differential, pressure ports are located upstream and downstream of the resistive element. The pressure port may be a simple access point that allows the pressure wave from the patient's breath to interact with the pressure transducer, thus enabling the pressure signal to be recorded. In one example of a typical mouthpiece, an upstream pressure port lies within the mouthpiece and a downstream port is also within the tube but on the opposite side of the resistive element from the upstream port. In another typical mouthpiece, however, the downstream port may be located outside the tube, measuring atmospheric pressure instead of pressure within the tube. In fact, the downstream port may be non-existent wherein the spirometry circuitry assumes the downstream pressure, had it been actually measured, would be equivalent to atmospheric pressure.

Several factors should be considered to ensure reliable pressure readings are obtained. For instance, the upstream pressure port preferably should be exposed to laminar air flow which, simply put, entails relatively organized flow (i.e., not turbulent flow) with limited “eddies” in the flow stream. This pressure port positioning increases the chance that the upstream port will measure pressure that is representative of the majority of air flow and not just a “whirlpool” of flow which could have a different pressure. Consequently, mouthpieces and resistive elements preferably should be designed to provide laminar flow over the upstream port. As another way for obtaining reliable pressure readings, mouthpieces may be individually calibrated to ensure a pressure measured on a first tube may be compared against normative values that were obtained on another tube. These calibration measures guard against the inevitable variability that exists within testing equipment due to manufacturing tolerances and the like. For example, an engineer may design a mesh resistive element to produce a designated level of resistance to air flow but the manufacturer may make, a first resistive element with slightly less resistance than the designated resistance and a second resistive element with slightly more resistance than the designated resistance. Without calibrating these “imperfect” resistive elements, a physician would have difficulty comparing a patient's breath tests performed on the two different mouthpieces. Design and calibration of such mouthpieces and resistive elements is well known to those of ordinary skill in the art.

Thus far, common mouthpieces have been described. More specialized mouthpieces do, however, exist. For example, several mouthpieces, such as those described in U.S. Pat. Nos. 3,621,833 and 4,991,591, possess limited variable resistive characteristics. Such a resistive element might entail a moveable plug that suddenly advances into an orifice of a mouthpiece precluding any air flow. Doing so may help a medical practitioner evaluate, for example, alveolar lung pressure. These variable resistive elements provide, however, only limited degrees of air flow obstruction such as relatively unimpeded flow (i.e., “open configuration”), whereby the plug is not positioned within or across an opening in the mouthpiece, and absolutely no flow (i.e., “closed configuration”) whereby the plug is positioned across an opening precluding substantially any air flow. These resistive elements do not provide a “partially open” configuration, thereby allowing limited air flow, that can be maintained in a static position long enough to obtain physiological measurements.

Other examples of prior art resistive elements may provide a partially open configuration that allows varied amounts of resistance to air flow but these same devices do not provide, for example, a completely closed orientation which delivers “infinite” or total resistance.

The prior art's shortcomings are critical because certain breathing tests require a closed configuration while other tests require partially open or substantially open configurations that provide smaller resistances to air flow. Also, the prior art designs are overly complex and expensive to manufacture because they may involve expensive electronic circuitry that determines an exact moment in time for moving a plug into an orifice of a mouthpiece. Such a feature is unnecessary for many HRV studies. Consequently, the mouthpieces and equipment needed to operate the mouthpieces make the use of, for example, disposable mouthpieces, cost prohibitive. This high cost may lead to many patients not being evaluated in countries where medical resources are limited. In addition, the complexity of the prior art devices raises a barrier to non-specialized physicians who cannot take the time to learn how to use the overly complex devices. In the end, tests that rely on the prior art mouthpieces may not be performed as often as should be the case. Consequently, many patients develop illnesses that could have been managed or prevented had the malady been diagnosed at early onset through, for example, an HRV study.

Therefore, a need exists for an affordable and non-complex mouthpiece which provides varying levels of air flow resistance with open and closed orientations, as well as orientations therebetween. A need also exists for a mouthpiece that maintains laminar flow characteristics in these varying orientations.

### SUMMARY OF THE INVENTION

The invention entails a novel but non-complex mouthpiece that a patient may blow into during a breathing test. The breathing tests may be conducted pursuant to, for example, HRV or general spirometric testing. The mouthpiece is for use in a spirometer and can be manufactured affordably. The mouthpiece provides varying levels of air flow resistance to the patient's breathing by providing open and closed orientations as well as orientations therebetween. The mouthpiece maintains laminar flow characteristics in the varied orientations.

In one embodiment of the invention, the mouthpiece comprises a tube forming a conduit between an upstream end and a substantially closed downstream end. An opening is formed through the tube and is proximate to the downstream end of the tube. The mouthpiece also comprises a resistive element that is positioned substantially across the tube opening. In addition, the mouthpiece may comprise an outer sleeve that is slid along the exterior of the tube. The outer sleeve may be slid along the tube thereby covering portions of the tube opening in varying amounts. Thus, the outer sleeve may be slid into one position wherein the tube opening is uncovered and a small amount of resistance is encountered by the patient. The outer sleeve may then be advanced into a closed position wherein the tube opening is substantially sealed and the patient experiences a large resistance to his breathing. Many partially closed positions may also exist between the open and closed positions thereby allowing for variable amounts of resistance to air flow.

To clearly indicate to the user how far the outer sleeve has been advanced, indicia, such as markings, may be made on the outer sleeve or tube indicating, for example, the first tube opening is fifty; percent occluded. An alternative embodiment of the invention may use a slidable inner sleeve which may be slid across the opening of a tube to provide varying levels of resistance to air flow. Another embodiment of the invention incorporates a slidable resistive element that may be slid within the main tube until an opening in the main tube is completely occluded. The incremental closure of the tube opening provides for variable resistances to air flow within the tube. Consequently, the present invention allows one mouthpiece to be used for a variety of different physiological tests that require varying levels of air flow resistance. Thus, the mouthpiece of the present invention provides advantages in convenience, ease of use and affordability.

The foregoing has outlined rather broadly the features of the present invention in order that the detailed description of the invention that follows may be better understood. Additional features and advantages of the invention will be described hereinafter which form the subject of the claims of the invention.

### BRIEF DESCRIPTION OF THE DRAWINGS

The present invention may be better understood, and its numerous objects, features and advantages made apparent to those of ordinary skill in the art by referencing the accompanying drawings, which illustrate, by way of example, embodiments of the invention. The use of the same reference number throughout the several figures designates a like or similar element however not all similar elements use the same reference number.

FIG. 1 is an example embodiment of the invention that illustrates a top front longitudinal section view of a mouthpiece, for use in a spirometer, with a slidable outer sleeve.

FIGS. 2A-2C are example embodiments of the invention that illustrate top longitudinal views of a mouthpiece, for use in a spirometer, with a slidable outer sleeve.

FIG. 3 is an example embodiment of the invention that illustrates a top front longitudinal section view of a mouthpiece, for use in a spirometer, with a slidable inner sleeve.

FIG. 4 is an example embodiment of the invention that illustrates a top longitudinal view of a mouthpiece, for use in a spirometer, with a slidable inner sleeve.

FIGS. 5A-5B are example embodiments of the invention that illustrate top longitudinal views of a mouthpiece, for use in a spirometer, with an outer tube that slides over an inner tube.

FIGS. 6A-6B are example embodiments of the invention that illustrate a top front longitudinal section view of a mouthpiece, for use in a spirometer, with a slidable resistive element.

FIG. 7 is an example embodiment of the invention that illustrates a top front longitudinal section view of a mouthpiece assembly, for use in a spirometer, with a tube and slidable caps.

FIG. 8 is an example embodiment of the invention that illustrates top front longitudinal section views of a mouthpiece assembly, for use in a spirometer, with a tube and slidable plugs.

FIG. 9 is an example embodiment of the invention that illustrates a longitudinal side section view of a mouthpiece, for use in a spirometer, with a slidable section.

FIG. 10A is an example embodiment of the invention that illustrates a top front longitudinal section view of a mouthpiece, for use in a spirometer, with an insertable, obstructive disk.

FIGS. 10B and 10C are example embodiments of the invention that illustrate a front view of an obstructive disk for use in a spirometric mouthpiece.

FIG. 10D is an example embodiment of the invention that illustrates a side view of a mouthpiece, for use in a spirometer, with a slot for receiving an insertable, obstructive disk.

### DETAILED DESCRIPTION

Slidable Outer Sleeve

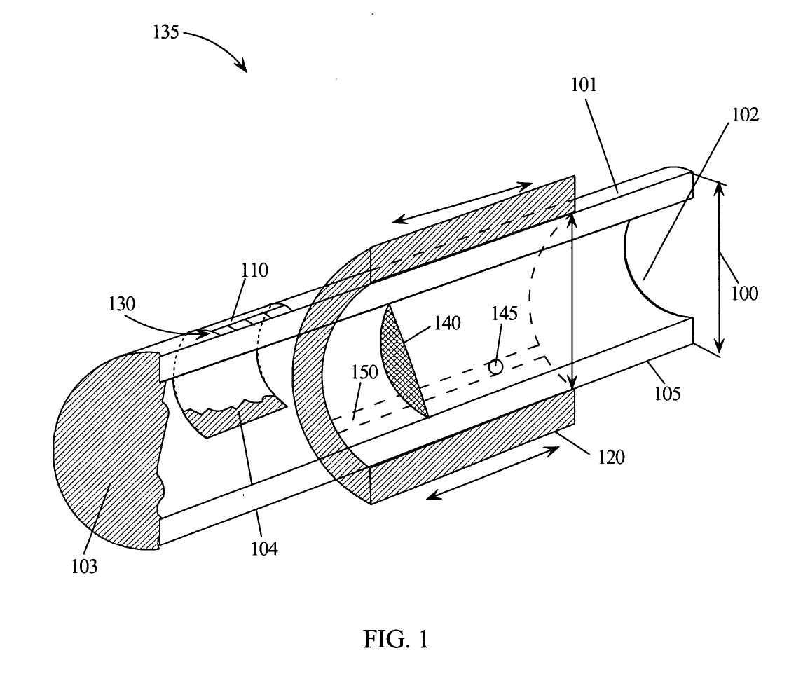

As illustrated in FIG. 1, one example embodiment of the invention generally entails a spirometric mouthpiece or breathing tube 135 comprising a first tube 101 with an upstream or distal end 102 that a patient may blow into. The tube 101 also has a downstream or proximal end 103. The tube 101 may have one or more openings 110 formed within or through the tube wall 105. The opening 110 may be located near the upstream end 102 or downstream end 103 or anywhere in-between. In addition, the downstream end 103 may be open or closed.

The one or more openings may be covered with porous fabric 104 to provide resistance to air flow thereby constituting a resistive element. When the patient blows into the mouthpiece, the porous fabric 104 resistive element cooperates with the tube 101 to provide a conduit for the patients' air while providing resistance to the air flow so that the air pressure can be generated and recorded.

The mouthpiece may also include a movable second tube or outer sleeve 120 that is slidably connected along the exterior of the first tube 101. The outer sleeve 120 may be moved or slid, in a telescoping manner, over the first tube 101 in order to obstruct the openings 110 of the first tube 101 in varying degrees. The inner diameter of the outer sleeve 125 may be sized such that the outer sleeve 120 slides, with some resistance, over the outer diameter 100 of the first tube 101.

As used herein, the terms “slide” or “slidable”, or variations thereof, should not be limited to: define an action that entails smooth continuous motion. Using the present embodiment as an example, slide or slidably may describe movement, advancement, re-positioning or a change of position of the outer sleeve 120 over the first tube 101. The, change of position may occur in numerous degrees of gradation. For example, the outer sleeve 120 may advance in 1 nanometer increments or in 15 mm increments.

The resistance to this change of position may also be implemented in various 10 forms. For example, as the outer sleeve 120 slides over the tube 101, the clinician may encounter resistance due to the inner diameter 125 of the outer sleeve 120 being only slightly larger than the outer diameter 100 of the tube 101. As another example, the outer sleeve 120 may cooperate with the tube 101 in a manner similar to how a screw cooperates with a nut. The outer sleeve 120 may have spiral grooving along its inner surface that cooperates threads located on the outer surface of the tube 101. The grooves and threads may cooperate with one another so that applied force acts in a spiral path along the grooves while the resisting force acts along the axis of the tube 101. Another embodiment of the invention may entail a ridge on the inner surface of the outer sleeve 120 that cooperates, in an elastic fashion, with grooves, indentions or recesses on the outer surface of the tube 101. As the outer sleeve is advanced or slid over the opening 510, the ridge may “click” as it advances into a corresponding recess on the tube 101

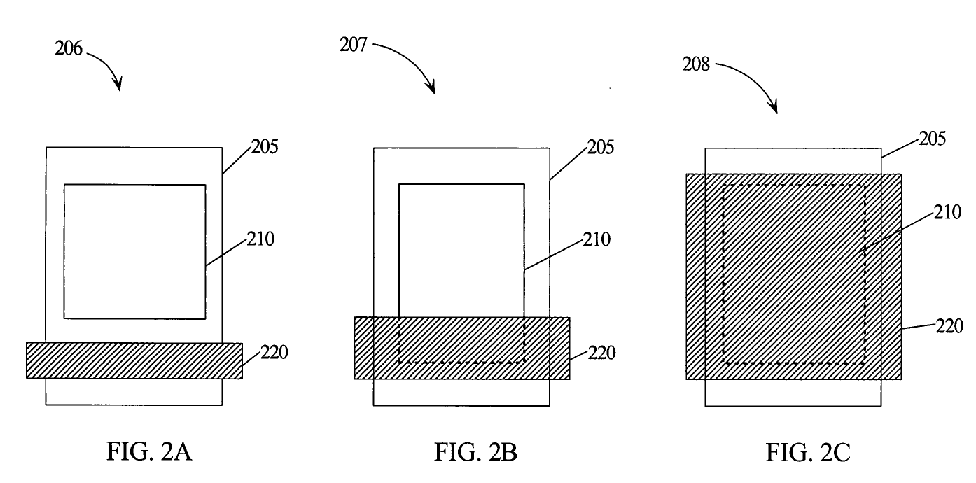

As the outer sleeve 120 is selectively slid into a range of different positions, various degrees of opening 110 occlusion are created. For example, in the “closed position” 208, as illustrated in FIG. 2C, or “0% open position”, the opening 210 is obstructed or covered by the outer sleeve 220, providing no output for expired air. In the “open position” 206, as illustrated in FIG. 2A, or “100% open position”, the opening 210 is unobstructed or uncovered by the outer sleeve or second tube 220. Varying degrees of opening 210 obstruction exist between the open and closed positions. As illustrated in FIG. 2B, the “75% open” position obstructs only a portion of the tube opening 210. More precisely, 25% of the opening's 210 surface area is covered providing for limited air flow through the opening 210. This orientation may be maintained throughout the breathing study. The “25% open” position may obstruct 75% of the opening 210 surface area allowing for decreased air flow as compared to the “75% open” position.

When a patient blows into a mouthpiece of the present invention, the degree of opening 210 obstruction determines, to an extent, the resultant air pressure. For example, in HRV testing the Valsalva test is performed when a patient exhales into a closed breathing tube. The closed orientation helps the patient reach the 40 mm Hg of pressure for 15 seconds that the test requires. Breathing in this manner has been shown to be helpful in assessing the ANS because such breathing will cause heart rate fluctuations in a patient with normal autonomic nervous system function. Thus, the closed tube configuration 208 of the present invention could be used for this test.

In contrast, the Slow Metronomic Breathing test does not require a prolonged 40 mm Hg pressure. Instead, during the test the patient breathes deeply and evenly at six breaths per minute. Consequently, a closed tube configuration 208 would not work. This breathing frequency has been shown to properly tax the respiratory system producing heart rate fluctuations in individuals with normal autonomic nervous systems. One reliable method used to gauge patient compliance with the six breathes/minute regimen entails monitoring the patient's air flow, volume and/or pressure in conjunction with ECG measurements. By using a spirometer, a physician can monitor the peaks and troughs of a patient's respiration cycle and then check to ensure there are, for example, six air pressure peaks per minute. The present invention's open tube configuration 206, or partially open configuration 207, would suffice for the Metronomic test because the patient would be able to breath in and out through the opening 210 but could still register an air pressure for tracking respiration. The aforementioned HRV tests may be conducted using standard HRV testing equipment and methods known to those of ordinary skill in the art (e.g., using the Task Force Report for Heart Rate Variability: Standards of Measurement, Physiological Interpretation, and Clinical Use, Circulation Vol. 93. No 5, 1996). One of ordinary skill in the art will appreciate that the invention is suitable for many types of breathing tests including, as examples, HRV testing and general spirometric testing.

As seen in FIG. 1, When tracking respiration in any test, a pressure transducer requires a threshold pressure to “pick up” a signal. If a patient fails to produce such a threshold pressure, the present invention allows the second tube or outer sleeve 120 to be incrementally slid over the opening 110 of the first tube 101 until the threshold pressure is achieved. For example, a small child or small adult may require a 50% closed orientation to provide the necessary threshold pressure whereas a large adult may be able to perform the Metronomic test best at a 100% open orientation 106. In short, the exemplar tube could function for both the Valsalva and the Metronomic tests for a variety of patients, thus providing convenience for the physician as well as cost savings.

Varying the air that flows through opening 110 has further advantages. For example, some patients have trouble exhaling to the point of complying with the Metronomic breathing protocol. Asthmatics or individuals afflicted with chronic obstructive pulmonary disease (COPD) must combat “air trap” when breathing deeply as required by the Metronomic protocol. “Air trap” occurs because the asthmatic can inhale with little difficulty but has difficult exhaling. The result produces “trapped air” within the lungs which further frustrates the patient's ability to exhale. To facilitate deep expiration, continuous positive airway pressure (CPAP) therapy is used to create a back pressure of a certain threshold. While a fully open orientation 206 may not meet this threshold, a partially closed orientation 207, for example 50% closed, may provide the needed pressure while still allowing the patient to breath throughout the minute long procedure required by the Metronomic study.

Furthermore, the invention may facilitate tests for measuring baroreflex sensitivity. Such measurements may be important because, for example, low baroreceptor sensitivity is associated with a higher risk of cardiovascular disease, including sudden cardiac death. During the breathing test, a partially open orientation 207, for example 50% open, would create extra airway pressure, as opposed to a 100% open configuration 206, while still allowing multiple breathing cycles to occur. The same could not be said for tube in a closed orientation 208. This increase in air pressure would cause a respective change in intrathoracic pressure. The change in pressure would in turn irritate baroreceptors in the circulatory system's aortic arch. The irritation would result in a change in heart rate for those with normal baroreceptor sensitivity. In short, the novel mouthpiece would allow for measurement of HRV caused by deep breathing at, for example, 50% and 100% open 206 orientations, so that critical comparisons in baroreceptor sensitivity could be made.

The air resistance provided by the mesh fabric 104 resistive element, which may be placed over the opening 110 of the embodiment illustrated in FIG. 1, may be augmented with, or substituted for, another resistive element placed within the tube 101. For example, a porous disk 140 could serve as a resistive element located within the tube 105. The mesh fabric 104 placed over the opening 110 may serve as a resistive element by providing resistance to air flow. The porous disk 140 may also provide resistance to air flow and is likewise a resistive element. One of ordinary skill in the art will recognize that the porous disk 140 and opening 110 with mesh fabric 104 may be located in any number of positions associated with the invention, and they will continue to function as resistive elements as long as they provide resistance to air flow. For example, the porous disk 140 may exist within the tube 101 and be spaced equidistant between the upstream end 102 and downstream end 103 of the tube 101. In the alternative, the porous disk 140 resistive element may be located at or near either end 102, 103 of the tube 101. In addition, the opening 110 and porous mesh 104 may be spaced equidistant between the two ends 102, 103 of the tube. The porous mesh 104 may be affixed to the inside or the outside of the tube 101.

A pressure port 145 could be located upstream of the porous disk 140 using methods commonly known to those of ordinary skill in the art. The second tube 120 could have a channel 150 that would ensure the outer sleeve 120 could be slid over the tube 101 without obstructing any tubing connected to the pressure port 145.

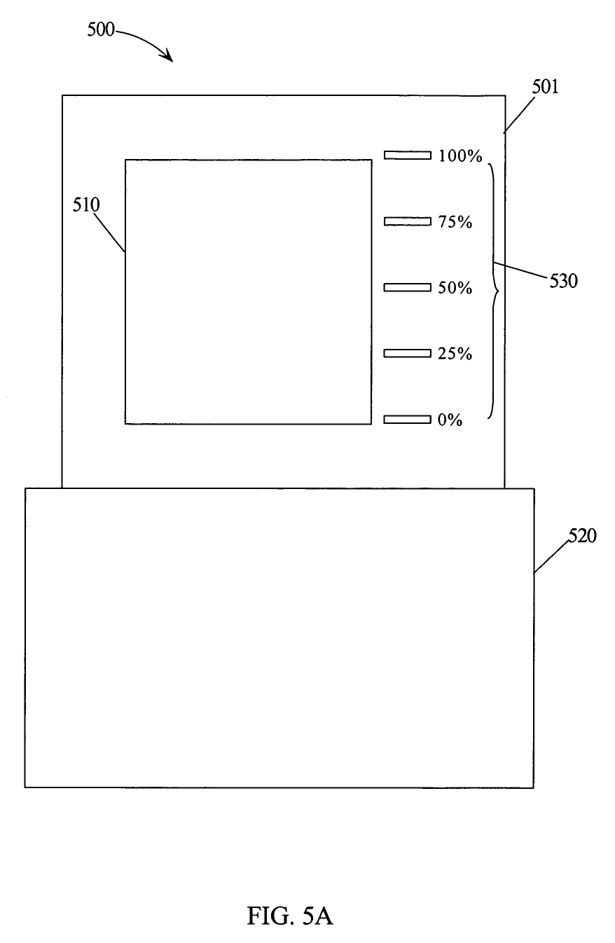

Calibration for the tube 101 may be performed according to different closure configurations. In one embodiment of the invention, five separate calibration values may be provided for the 0% open 108, 25% open, 50% open, 75% open and 100% open 206 configurations, thus ensuring air flow readings taken in each orientation may be compared with normative values. These percentiles could be marked as a display 130 on the tube 105 to indicate when the tube opening is uncovered 206, partially covered 207 or substantially sealed 208. The display 130, which may be graduated, could indicate to the physician, for example, that the tube opening 110 is 50% occluded and that the calibration value for a 50% closure should be used in calculating air flow values. An embodiment of a display incorporating indicia 130 is illustrated in FIG. 5A which depicts an “open position” configuration 206 where the outer tube 520 does not obstruct the opening 510 of the inner tube 501. A “partially closed” position 207 is illustrated in FIG. 5B where the outer sleeve 520 has been slid over the opening 510 until it reached the marking designated “25%” 521, which is indicative of 25% obstruction of the opening 510.

Advancing the outer tube 520 to a precise location may be very important in some testing situations. For example, some calibration methods used to calibrate the tube 505 may have small tolerances. More specifically, a calibration value calculated for a 25% obstruction of the opening 510 may not be accurate for a 28% obstruction of the opening 510. Consequently, the indicia 530 may include a means for precisely indicating the level of opening 510 obstruction. The indicia 530 may cooperate with an obstructive element to achieve the desired level of opening 510 obstruction. In one embodiment of the invention, the outer sleeve 520 may cooperate with the tube 505 in a manner similar to how a screw cooperates with a nut. The outer sleeve 520 may have spiral grooving along its inner surface that cooperates with threads located on the outer surface of the tube 505. The grooves and threads may cooperate with one another so that applied force acts in a spiral path along the grooves while the resisting force acts along the axis of the tube 505. Another embodiment of the invention may entail a ridge on the inner surface of the outer sleeve 520 that cooperates, in an elastic fashion, with grooves, indentions or recesses on the outer surface of the tube 505. As the outer sleeve is advanced or slid over the opening 510 the ridge may “click” as it advances into a corresponding recess on the tube 505. The recesses may be positioned so that the outer sleeve 520 precisely obstructs 25% of the opening 510 or 75% of the opening 510. To further help the clinician advance the outer sleeve to a proper level of opening 510 obstruction, an auditory stimulus, such as a “click”, may be provided when the outer sleeve 520 ridge advances into a recess. The movement of the ridge into the recess may also provide tactile stimulus to the user so that she or he understands the outer sleeve 520 is in proper position. Other embodiments of the invention may entail, for example, LED's or lights that illuminate when the outer sleeve 520 has been advanced to a certain point such as 25% closure of the opening 510. One of ordinary skill in the art will recognize that the indicia 530 may take the form of markings, display lights, LED's, color-coded bars, LCD's and other means that provide visual, tactile or auditory stimulus to the clinician to help the clinician appreciate the location of the outer sleeve 520.

Slidable Inner Sleeve

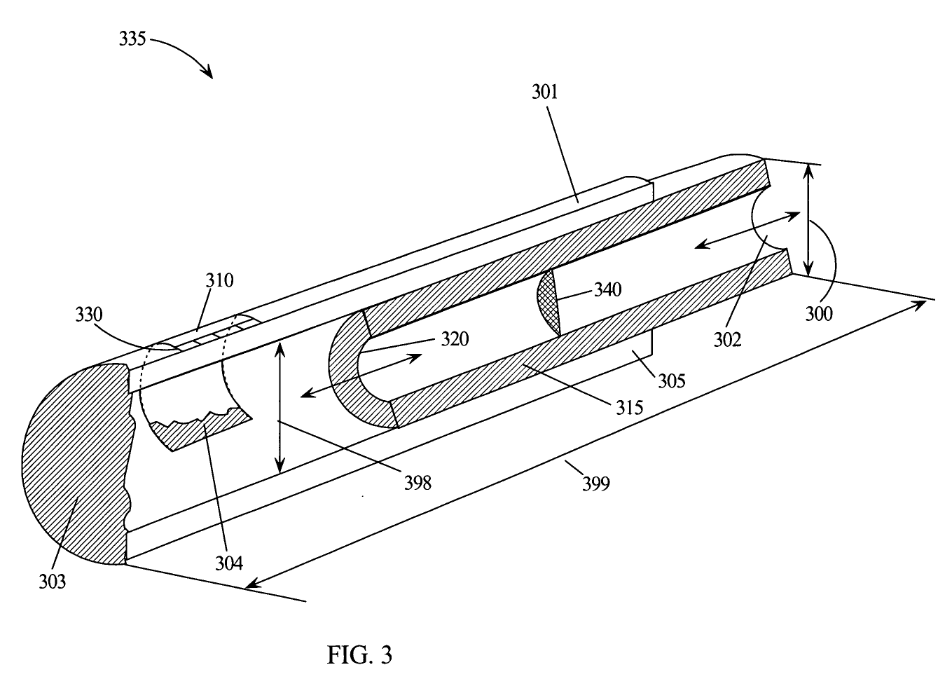

As illustrated in FIG. 3, an alternative embodiment of the invention generally entails a spirometric mouthpiece or breathing tube 335 comprising a first tube 301 with an upstream or distal end 302 that a patient may blow into. The tube 301 also has a downstream or proximal end 303. The tube 301 may have one or more openings 310 formed within or through the tube wall 305. The opening 310 may be located near the upstream end 302 or downstream end 303 or anywhere in-between. In addition, the downstream end 303 may be open or closed.

The one or more openings 310 may be covered with porous fabric 304 to provide resistance to air flow thereby constituting a resistive element. When the patient blows into the mouthpiece, the porous fabric 304 resistive element cooperates with the tube 301 to provide a conduit for the patients' air while providing resistance so that the air flow air pressure can be generated and recorded.

The mouthpiece may also include a movable second tube or inner sleeve 315 that is slidably connected along the interior of the first tube 301. The inner sleeve 315 may be moved or slid, in a telescoping manner, within the first tube 301 in order to obstruct the openings 310 of the first tube 301 in varying degrees. The outer diameter of the inner sleeve 300 may be sized such that the inner sleeve 315 slides, with some resistance, within the first tube 301 having an inner diameter 305.

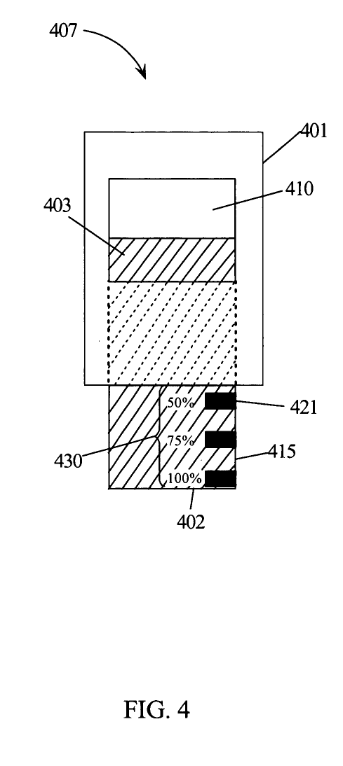

As the inner sleeve 315 is selectively slid into a range of different positions, various degrees of opening 310 occlusion are created. For example, in the “closed position,”or “0% open position,” the opening 310 is obstructed or covered by the inner sleeve 315, providing no output for expired air. In the “open position,” or “100% open position,” the opening 310 is unobstructed or uncovered by the inner sleeve or second tube 315. Varying degrees of opening 310 obstruction exist between the open and closed positions. As illustrated in FIG. 4, the partially open position. 407, for example “75% open”, obstructs only a portion of the tube opening 410. More precisely, 25% of the opening's 410 surface area is covered providing for limited air flow through the opening 410. This orientation may be maintained throughout the breathing study. The “25% open” position may obstruct 75% of the opening 410 surface area, allowing for decreased air flow as compared to the “75% open” position.

Again referring to FIG. 3, in the closed position, wherein the inner sleeve 315 completely obstructs the opening 310, the inner sleeve 315 may rest substantially within the main tube 301 allowing no portion of the inner sleeve 315 to extend from the tube 301. In the open position wherein the inner sleeve or second tube 315 does not obstruct the opening 310, however, there may be a portion of the inner sleeve 315 that extends from the first tube 301. As a consequence, in one example of the mouthpiece, the overall length of the tract 399 that air must pass though is increased. The ability to adjust the overall length of this tract 399 may promote laminar flow and, consequently, accurate respiration flow measurements. In addition, the downstream end 320 of the inner sleeve 315 may be tapered to facilitate laminar flow. FIG. 4 shows inner sleeve 415 obstructing approximately half of opening 410 of the first tube 401. The “50%” marking 421 constitutes part of display 430 which indicates when the tube opening 410 is uncovered or substantially sealed by the inner sleeve 315. The display 415 is located on the inner sleeve 415 and indicates a “half closed” configuration whereby a portion of the inner sleeve 415 extends from the first tube 401.

The air resistance provided by the mesh fabric 304 resistive element, which may be placed over the opening 310 of the embodiment illustrated in FIG. 3, may be augmented with, or substituted for, another resistive element placed within the tube 301. For example, a porous disk 340 could serve as a resistive element located within the tube 105 or within the inner sleeve 315. The mesh fabric 304 placed over the opening 310 may serve as a resistive element by providing resistance to air flow. The porous disk 340 may also provide resistance to air flow and is likewise a resistive element. One of ordinary skill in the art will recognize that the porous disk 340 and opening 310 with mesh fabric 304 may be located in any number of positions associated with the invention, and they will continue to function as resistive elements as long as they provide resistance to air flow. In addition, the opening 310 and porous mesh 304 may be spaced equidistant between the two ends 302, 303 of the tube. The porous mesh 304 may be affixed to the inside or outside of the tube 101.

Calibration for the mouthpiece 335 may be performed according to different closure configurations. For example, a different calibration value may be provided for the 0% open, 25% open, 50% open, 75% open and 100% open configurations to ensure air flow readings taken with different closure orientations may be compared with normative values. The level of closure may be marked on the tube 415 or inner sleeve, in a display 430, to ensure the user appreciates, for example, that the tube is 50% occluded and that the calibration value for a 50% closure should be used in calculating air flow values. These markings could also be placed on the upstream end 402 or downstream end 403 (and viewed through the opening 410) of the first tube 401. One of ordinary skill in the art will recognize that the indicia may take the form of markings, display lights, LED's, color-coded bars, LCD's and ridges or indentions that cooperate with a moving element, such as an adjustable outer sleeve, to provide auditory stimulus when the moving element is advanced to a certain ridge or indention. Various related embodiments were previously described above in the discussion related to the movable outer sleeve 520 embodiment of the invention.

Slidable Resistive Element

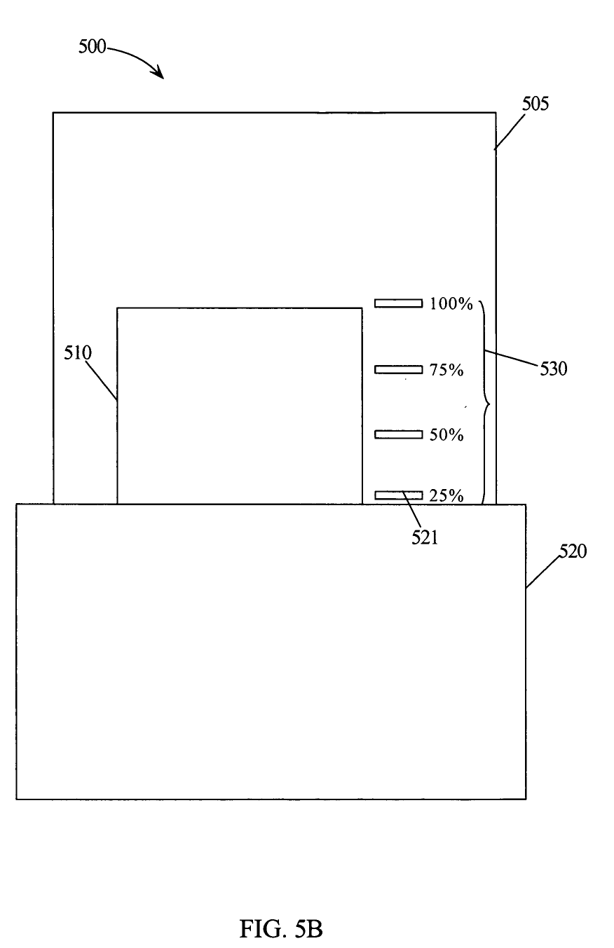

In another embodiment of the invention, as illustrated in FIG. 6A, a plug 605 slides within the main breathing tube 601 which has an upstream or distal end 602 and a downstream or proximal end 603. The tube 601 may have an opening 610 formed through the tube 601. The plug 605 is slidably connected along the interior of the tube 601. The plug 605 increases resistance to air flow, thus functioning as a resistive element, and shunts the air flow through the opening 610, which also provides resistance to air flow, when the plug 605 is positioned near the opening 610. In the closed position the plug 605 may rest across the opening 610 or upstream of the opening 610 thus preventing substantially any air flow through the opening 610 and substantially sealing the tube 601. In the open position, the plug 605 is pulled back towards the downstream end 603 of the tube 601 and is situated downstream from the opening 610. A pressure port may be located upstream or downstream of the opening 610 or even, for example, within the plug 615. This plug configuration may also be used in the previously described embodiments of the mouthpiece where the plug may or may not be movable depending on the designer's choice. As seen in FIG. 6B, partial levels of closure (e.g., the plug occludes 50% of opening 300) and corresponding calibration values, as described above, are available with this embodiment of the invention. Also, as described above, a display 630 may be disposed on the plug 605 or tube 601 to indicate when the opening 610 is uncovered or substantially sealed by the plug 605. A handle 603 may be attached to plug 615 to facilitate, sliding the plug 615 within the tube 601.

### Other Embodiments

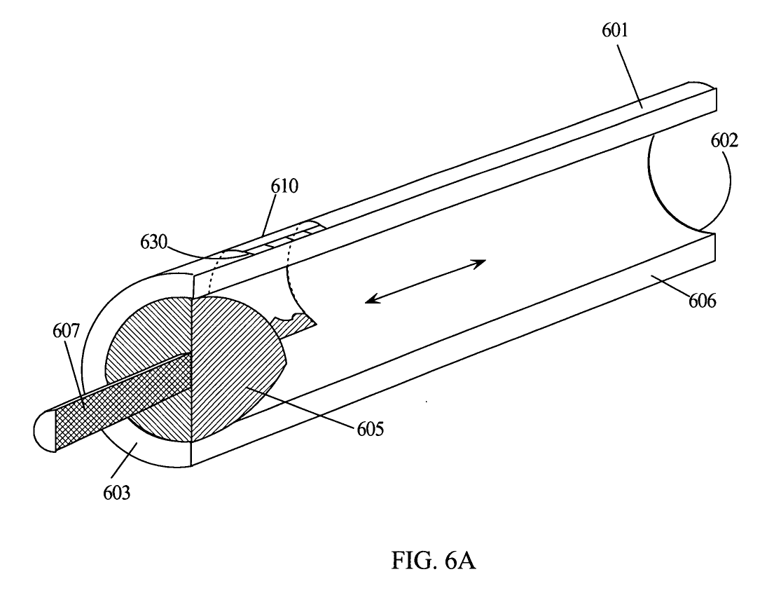

In another embodiment of the invention, as illustrated in FIG. 7, the mouthpiece assembly 700 comprises a tube 701 forming, a conduit between an upstream opening or distal end 715 and a downstream opening or proximal end 710. The assembly may also have a first cap 730 that may be slidably connected along the exterior of the tube 701 wherein the first cap 730 may be slid over the downstream opening of the tube 710. The first cap 730 may have a substantially closed base 740 that defines an opening 745 with a diameter 750 that is, smaller than the diameter 725 associated with the downstream opening 710 of the tube. The difference in diameters provides for a partially closed configuration that provides decreased air flow and increased resistance when compared to tube 701 used without the cap 730 (open orientation). The assembly 700 may also include a second cap 755 that may be slidably connected along the exterior of the tube 701. The second cap 755 may be slid over the downstream opening of the tube 710. The second cap 755 may contain a closed base 790 that substantially seals the downstream opening 710 of the tube 701 resulting in a closed orientation.

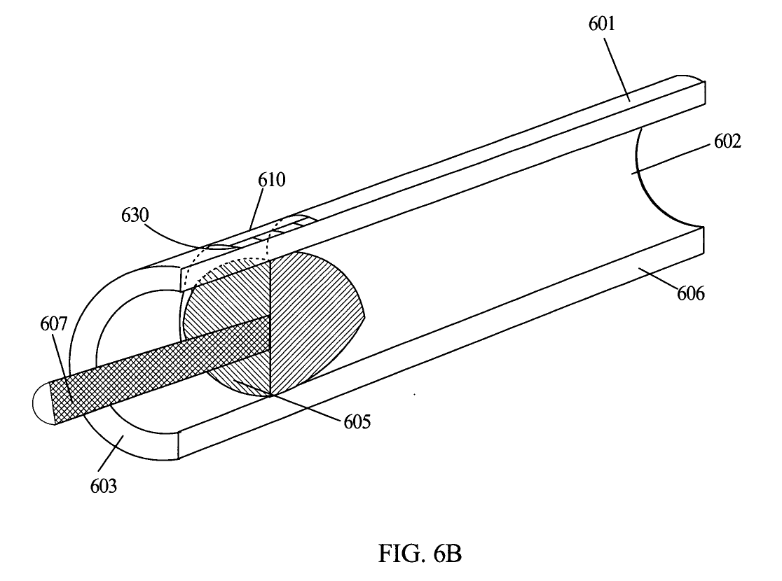

In an embodiment similar to the embodiment represented in FIG. 7, FIG. 8 illustrates a mouthpiece assembly 800 with a tube 801 forming a conduit between an upstream opening 820 and a downstream opening 825. The assembly 800 may include a first plug 830 that may be slidably connected along the interior of the tube 801 wherein the first plug 830 may be slid within the downstream opening 810 of the tube 801. The first plug 830 may comprise a substantially closed base 840 that defines an opening 845 which is smaller than the downstream opening 810 of the tube 810. The smaller diameter 850 provides a partially closed orientation and ensures an increased resistance to air flow than would be present in the tube 801 without the first plug 830 inserted within the downstream opening 825 (open orientation). The assembly 800 may also incorporate a second plug 855 that may be slidably connected along the interior of the tube 801. The second plug 855 may be slid within the downstream opening 825 of the tube 801. The second plug 855 may have a closed base 890 that substantially seals the downstream opening 825 of the tube. 801 when the second plug 855 is in use, thus resulting in a closed orientation.

Referring to FIGS. 7 and 8, the diameters 750 and 850 may be varied to provide varying levels of air resistance. Therefore, a mouthpiece assembly could be shipped to a physician with several caps, such as the embodiments shown in FIGS. 7 and 8, that provide for various levels of air resistance. The physician could then perform a variety of tests, such as the Valsalva and the Metronomic tests, using only one tube for the patient. The caps would be uncomplicated and cost-effective, thereby promoting proper testing of more patients.

The air resistance provided by the caps 730, 830 may be augmented or substituted for by placing, for example, a porous disk 140, as seen in FIG. 1A, within the tube 701, 801. In addition, openings could exist in the wall of the tube, as seen in element 110, that could be covered in varying degrees as the caps 730, 830, 755, 855 are slid across the openings 110. Furthermore, a resistive material, such as a mesh fabric, could be placed over the openings 110 or 745, 845. Thus, there are many forms of resistive elements that may be incorporated with the invention. These elements may be positioned in a number of orientations. For example, the porous disk 140 may be located at substantially either end of the tube 701, 801 or between the two ends 710, 810, 720, 820, and will continue to function as a resistive element as long as it provides resistance to air flow.

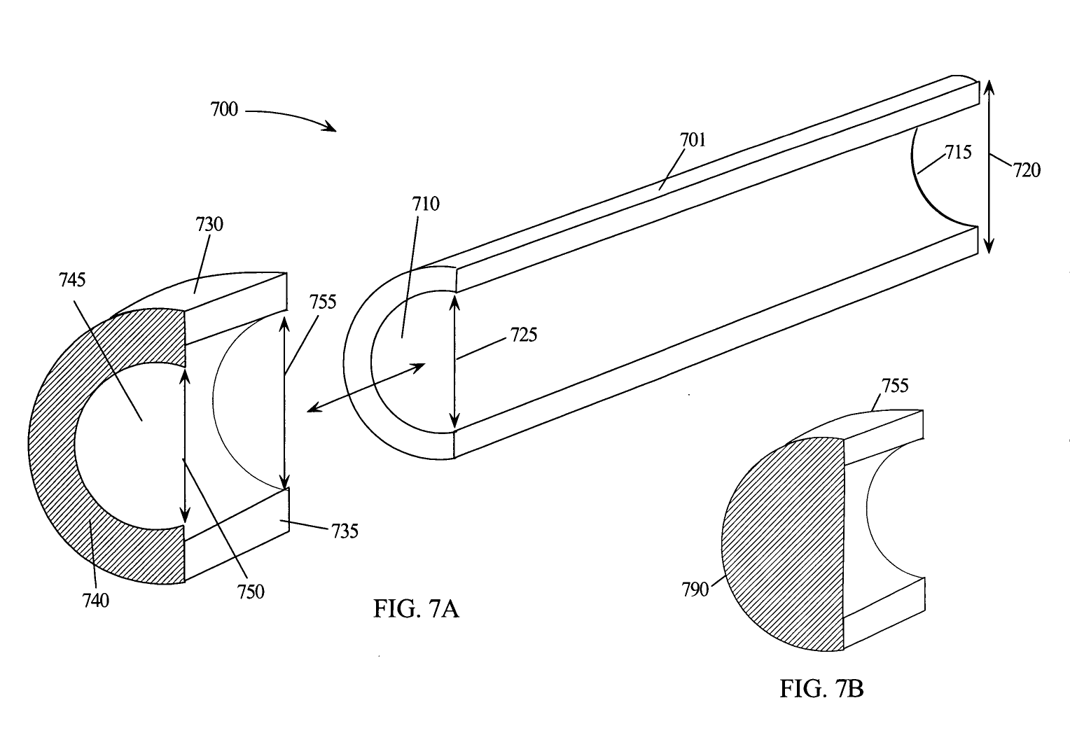

As illustrated in the previously described embodiments of the invention, the invention uses a variety of ways to create varying levels of resistance to air flow. For example, an alternative embodiment of the invention, as seen in FIG. 9, has a first opening 902 and a second opening 910 for a patient's breath to respectively enter and exit the mouthpiece 935. A resistive element may be advanced across or within the second opening 910, in incremental fashion, to permit varying degrees of obstruction of the second opening 910. The resistive element may entail a sleeve or section 920 disposed within the wall 905 of a tube 901 whereby the sleeve 920 may be advanced across the second opening 910. The sleeve 920 may incorporate a display 930 that indicates how far across the second opening 910 the sleeve 920 has been advanced. In alternative embodiments of the invention, the resistive, element may be a conical section that is advanced into (i.e., across) one of the tube openings whereby the tube opening is increasingly obstructed until the section contacts two points, for example, diametrically opposed to one another, wherein complete obstruction of the opening occurs.

In yet another embodiment of the invention, a film may be positioned along the outside of the tube 101. The film may roll up upon itself when the clinician desires no obstruction of the opening 110. The clinician may then unroll the film to selectively obstruct varying portions of the opening 110.

As seen above, there are various means for obstructing all or at least a portion of the second opening, wherein the means for obstructing can be configured to allow the second opening to be selectively obstructed. For example, the means for obstructing can be configured to provide for substantially no obstruction, some obstruction or substantially complete obstruction of an opening or outlet in the mouthpiece. The means for obstruction may be an outer sleeve, inner sleeve, slidable resistive element, cap, plug, film, or a section disposed within a tube wall.

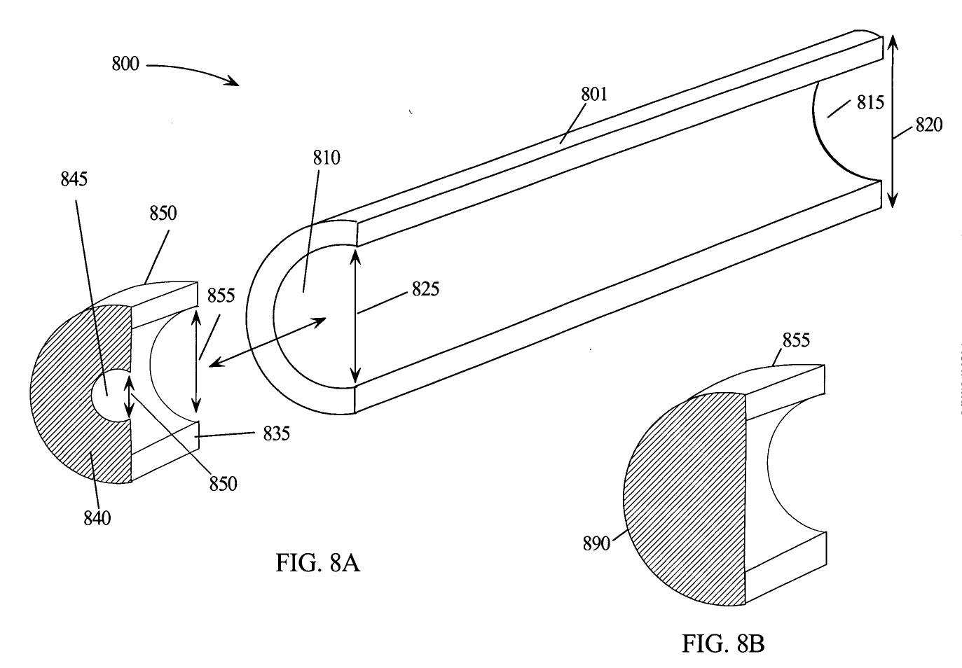

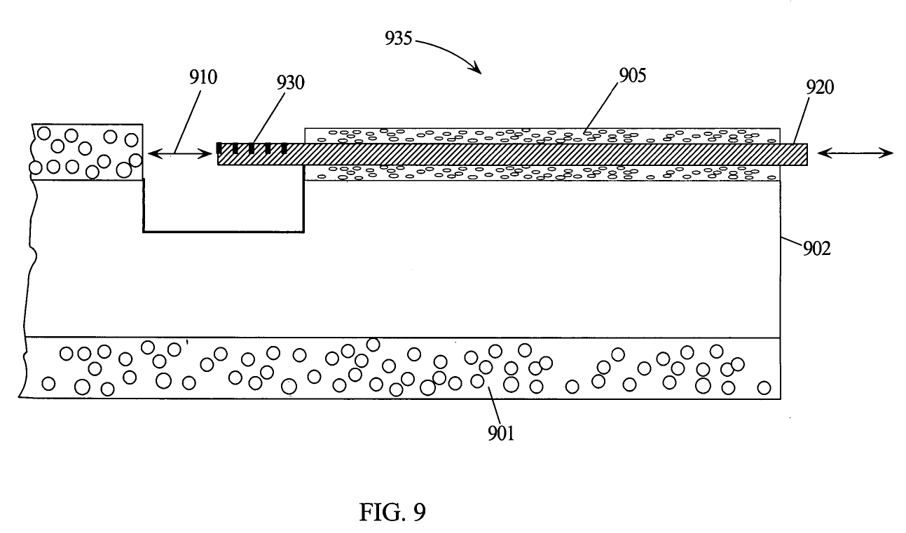

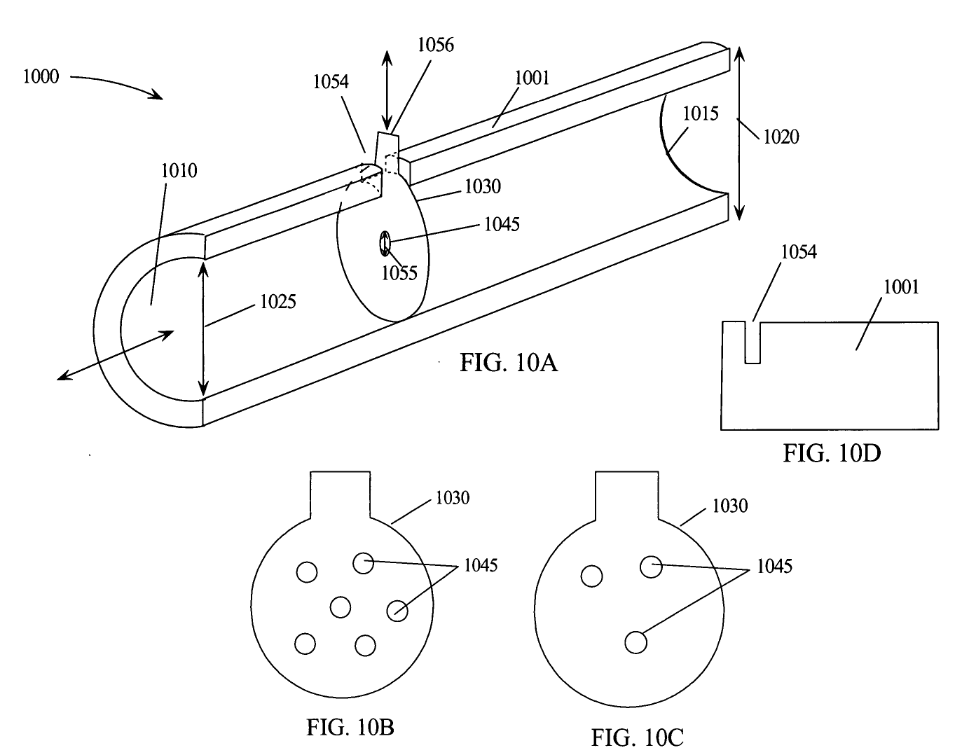

An additional example of a means for obstructing an opening is illustrated in FIG. 10A. A disk 1030 may be employed to selectively obstruct the opening 1010. Using handle 1056, the disk may be inserted into the tube 1001, through slot 1054 which exists in the wall of tube 1001 (FIG. 10C). Upon insertion of the disk 1030 into the tube 1001, the disk 1030 may substantially form a seal with the perimeter of the slot 1054. The disk 1030 may have an opening 1045 with a diameter 1055 that is smaller than the tube opening diameter 1025. Consequently, the disk 1030 will partially obstruct air flow to the opening 1010. A disk with no opening may substantially seal the opening 1010 so that substantially no air flow reaches or passes the opening 1010. A disk with an opening diameter 1055 substantially equal to opening, diameter 1025 would leave the opening 1010 substantially unobstructed. Consequently, a means for obstructing the opening 1010, such as a series of disks with openings 1045 of varying, diameters 1055, may be positioned to selectively obstruct various portions of the opening 1010. In one embodiment of the invention, as seen in FIGS. 10B and 10C, the disk 1030 may employ multiple openings 1045 to vary the level of opening 1010 obstruction. More precisely, disks 1030 with more openings 1045, as seen in FIG. 10B, may provide less obstruction of opening 1010 than a disk with fewer openings 1045, as seen in FIG. 10C.

As another example of a means for obstructing an opening of a tube, the tube 101 may comprise one or more removable panels or sections. The sections may exist as part of the tube wall or, for example, as part of a cap or disk placed within the tube or simply in cooperation with the tube. The clinician may remove one or more of the sections, thereby decreasing the degree of opening obstruction. The clinician can add or replace the sections to increase air flow obstruction.

One of ordinary skill in the art will appreciate that there are a number of other alternative embodiments available for implementing a means for obstructing an opening. The person of ordinary skill in the art will understand these alternative embodiments may allow for mouthpiece openings to be open, closed or partially obstructed, and that such embodiments are within the scope of the present invention.

All patents, publications and standards cited are incorporated by reference. Furthermore, it will be understood that certain of the above-described structures, functions and operations of the above-described preferred embodiments are not necessary to practice the present invention and are included in the description simply for completeness of an example embodiment or embodiments. In addition, it will be understood that specific structures, functions and operations set forth in the above-referenced patents and publications can be practiced in conjunction with the present invention, but they are not essential to its practice. It is therefore to be understood that within the scope of the claims, the invention may be practiced otherwise than as specifically described without actually departing from the spirit and scope of the present invention.

1. An apparatus for use in a spirometer comprising:
a first tube further comprising;
a proximal end;
a distal end;
a wall disposed between the distal end and the proximal end; and
an opening in the first tube wall, the opening having first and second points located along its perimeter;
a second tube further comprising a wall being slidably connected along the first tube wall;
a resistive element, that provides resistance to air flow, disposed substantially within the first tube wall;
wherein the second tube may be incrementally slid across the first tube opening in a telescoping manner from at least the first point on the first tube opening to at least the second point on the first tube opening to permit varying degrees of opening obstruction.

2. The apparatus of claim 1 wherein the first tube opening is substantially obstructed once the second tube is slid to the second point on the first tube opening.

3. The apparatus of claim 1 comprising an indicium that indicates how much of the first tube opening is obstructed by the second tube.

4. The apparatus of claim 1 wherein the resistive element is disposed substantially within the first tube.

5. The apparatus of claim 1 wherein the resistive element is disposed substantially across the first tube opening.

6. The apparatus of claim 1 wherein the resistive element is disposed substantially across the proximal end of the first tube.

7. The apparatus of claim 1 wherein the second tube wall is slidably connected along an inner surface of the first tube wall.

8. The apparatus of claim 1 wherein the second tube wall is slidably connected along an outer surface of the first tube wall.

9. An apparatus for use in a spirometer, the apparatus comprising:
a tube having a wall which forms a conduit between an upstream end and a substantially closed downstream end;
an opening formed through the tube wall that is proximate to the downstream end of the tube;
a resistive element positioned substantially across the tube wall opening; and
an outer sleeve slidably connected along the exterior of the tube, wherein the outer sleeve may be slid to selectively cover portions of the tube wall opening ranging from an open position wherein the tube wall opening is substantially uncovered to a closed position wherein the tube wall opening is substantially sealed.

10. The apparatus of claim 9 further comprising an indicium that indicates how much of the tube wall opening is covered by the outer sleeve.

11. An apparatus for use in a spirometer, the apparatus comprising:
a tube having a wall which forms a conduit between an upstream end of the tube and, a downstream end of the tube;
an opening formed through the tube wall wherein the conduit formed by the tube directs a patient's exhaled breath from the upstream end of the tube towards the tube wall opening;
a resistive element disposed substantially within the conduit tube so that the resistive element provides resistance to the patient's exhaled breath;
an outer sleeve slidably connected along~the exterior of the tube wall, wherein the outer sleeve may be slid to selectively cover portions of the tube wall opening ranging from an open position wherein the tube wall opening is substantially uncovered to a closed position wherein the tube wall opening is substantially sealed.

12. The apparatus of claim 11 further comprising an indicium that indicates how much of the tube wall opening is covered by the outer sleeve.

13. The apparatus of claim 11 wherein the resistive element is disposed substantially across the tube wall opening.

14. The apparatus of claim 11 wherein the resistance element is disposed substantially across the downstream end of the tube.

15. An apparatus for use in a spirometer, the apparatus comprising:
a tube having a wall which forms a conduit between an upstream end and a downstream end;
an opening formed through the tube wall wherein the conduit formed by the tube directs a patient's exhaled breath from the upstream end of the tube towards the tube wall opening;
a resistive-element disposed substantially within the conduit tube so that the resistive element provides resistance to the patient's exhaled breath;
an inner sleeve slidably connected along the interior of the tube, wherein the inner sleeve may be slid to selectively cover portions of the tube wall opening ranging from an open position wherein the tube wall opening is substantially uncovered to a closed position wherein the tube wall opening is substantially sealed.

16. The apparatus of claim 15 further comprising an indicium that indicates how much of the tube wall opening is covered by the outer sleeve.

17. The apparatus of claim 15 wherein the resistive element is disposed substantially across the tube wall opening.

18. The apparatus of claim 15 wherein the resistive element is disposed substantially across the downstream end of the tube.

19. The apparatus of claim 15 wherein at least a portion of the inner sleeve is not disposed entirely within the tube when the tube wall opening is substantially uncovered by the inner sleeve.

20. The apparatus of claim 15 wherein substantially no portion of the inner sleeve extends from the tube when the tube wall opening is substantially sealed by the inner sleeve.

21. An apparatus for use in a spirometer, the apparatus comprising:
a tube having a wall which forms a conduit between an upstream end of the tube and a downstream end of the tube;
an opening formed through the tube wall wherein the conduit formed by the tube directs a patient's exhaled breath from the upstream end of the tube towards the downstream end of the tube;
a resistive element slidably connected along the interior of the tube, wherein the resistive element may be slid to selectively cover portions of the tube wall opening ranging from an open position wherein the tube wall opening is uncovered to a closed position wherein the tube wall-opening is substantially sealed.

22. The apparatus of claim 21 further comprising an indicium that indicates how much of the tube wall opening is covered by the resistive element.

23. An apparatus for use in a spirometer, the apparatus comprising:
a tube having a wall which forms a conduit between an upstream end of the tube and a downstream end of the tube;
the tube comprising a first opening and a second opening wherein the conduit formed by the tube directs a patient's exhaled breath from the first opening towards the second opening; and
a means for obstructing all or at least a portion of the second opening, wherein the means for;obstructing can be configured to allow the second opening to be selectively obstructed ranging from an open position, wherein the second opening is substantially unobstructed, to a closed position, wherein the second opening is substantially obstructed.

24. The apparatus of claim 23 further comprising an indicium that indicates how much of second opening is covered by the means for covering the second opening.

25. The apparatus of claim 23 wherein the means for obstructing is positioned proximate to the first opening.

26. The apparatus of claim 23 wherein the means for obstructing is positioned proximate to the second opening.

27. The apparatus of claim 23 wherein the means for obstructing comprises one or more disks.

28. The apparatus of claim 23 wherein the means for obstructing comprises one or more panels.

29. The apparatus of claim 23 wherein the means for obstructing comprises a second tube.

30. An assembly for use in a spirometer, the apparatus comprising:
a tube forming a conduit between an upstream opening and a downstream opening;
a first cap that may be slidably connected along the exterior of the tube, wherein the first cap may be slid over the downstream opening :of the tube, the first cap further comprising a substantially closed base that defines an opening which is smaller than the downstream opening of the tube thereby providing resistance to air flow when the first cap is slid,over the downstream opening of the tube; and
a second cap that may be slidably connected along the exterior of the tube, wherein the second cap may be slid over the downstream opening of the tube, the second cap further comprising a closed base that substantially seals the downstream opening of the tube when the second cap is slid over the downstream opening of the tube.

31. An assembly for use in a spirometer, the apparatus comprising:
a tube forming a conduit between an upstream opening and a downstream opening;
a first plug that may be slidably connected along the interior of the tube wherein the first plug may be slid across the downstream opening of the tube, the first plug further comprising a substantially closed base that defines an opening which is smaller than the downstream opening of the tube thereby providing resistance to air flow when the first plug is slid over the downstream opening of the tube; and
a second plug that may be slidably connected along the interior of the tube wherein, the second plug may be slid across the downstream opening of the tube, the second plug further comprising a closed base that substantially seals the downstream opening of the tube when the second plug is slid over the downstream opening of the tube.

32. A method for conducting a breathing test to assess the autonomic function of a patient comprising the steps of:
determining a level of pressure required by the breathing test;
selectably sliding a resistive element of a mouthpiece into one of three or more positions in response to the level of pressure required by the breathing test; and
the patient exhaling his breath into the mouthpiece wherein the mouthpiece comprises the following:
a tube with an upstream opening and a downstream opening wherein the exhaled breath from the patient enters the tube through the upstream tube opening and flows towards the downstream tube opening; and
the resistive element of the mouthpiece being slidably engaged with the tube so that the resistive element may be slid into any one of the three or more positions wherein a first position of the three or more positions is an open position having the downstream tube opening substantially uncovered by the resistive element, a second position of the three or more positions is a closed position having the downstream tube opening substantially covered by the resistive element and a third position of the three or more positions is a partially closed position having the downstream tube opening partially covered by the resistive element;
wherein the selected one of the three positions operates to help the patient achieve the level of pressure required by the breathing test when the patient exhales his breath into the upstream opening of the mouthpiece.
